(19) United States
(12) Patent Application Publication
(10) Pub. No.: US 2008/0318252 A1
(43) Pub. Date: Dec. 25, 2008

(54) Rapid assay to test anti-cancer drugs under physiological conditions

(76) Inventor: Scott Kachlany, Bridgewater, NJ (US)

Correspondence Address: University of Medicine and Dentistry of New Jersey;Office of Patents and Licensing, One Worlds Fair Drive, Suite 2100, Somerset, NJ, 08873, US

(21) Appl. No.: 12/150,038

(22) Filed: Apr. 23, 2008

Related U.S. Application Data
(60) Provisional application No. 60/925,794, filed on Apr. 23, 2007.

(51) Int. Cl. G01N 33/53

(52) U.S. Cl. 435/7.21

(57) ABSTRACT

This invention relates to an assay that allows for the rapid determination of the activity of a given drug against leukemic cells either taken from a patient or derived from a cell line. The assay is performed in the presence of whole blood or serum.

This applications claims priority from provisional application Ser. No. 60/925,794, filed Apr. 25, 2007.

The research leading to the present invention was supported, at least in part, by a grant from the National Institutes of Health (NIH R01DE16133). Accordingly , the Government may have certain rights in the invention.

This invention relates to an assay that allows for the rapid determination of the activity of a given drug against leukemic cells either taken from a patient or derived from a cell line.

Each year, more than 60,500 people die of hematologic malignancies (leukemia, lymphoma, myeloma) with more than 110,000 new annual diagnoses in the US alone. Current treatment for these cancers includes the use of synthetic compounds that target the cell division process of nearly all cells of the body, not just the cancerous ones. Furthermore, a significant percentage of patients eventually show resistance to many of the drugs, thus rendering treatment largely ineffective. Indeed, there is an effort to identify agents that induce cancer cell death by methods other than damage to DNA or cell division (20).

The initial identification and testing of novel anti-cancer agents relies on in vitro killing assays using relevant cancer cell lines. While in vitro assays performed under cell culture conditions prove useful and necessary for preclinical testing of new therapeutics, extrapolation to the physiological conditions of a living organism is often difficult or impossible (27). Because of the high cost of drug development ($800 million), new drug screens are constantly being sought to more efficiently eliminate or identify candidate therapeutic agents (27). Indeed, increasing the clinical success rate from ⅕ to ⅓ because of more effective preclinical drug screens would reduce drug development costs by more than $200 million (27).

The activity, specificity, or toxicity of a compound in the physiological environment can vary significantly from cell culture conditions. While no in vitro assay or screen can represent the complexity of the human body, several assays have been developed to more closely mimic in vivo conditions. Several of these assays include the colony forming cell assay using bone marrow cells (27,29), hepatic drug biotransformation assays (3), and assays in whole blood (4,45). Because most chemotherapeutic agents are administered intravenously and are therefore immediately affected by blood cell components, screening for potential drugs in the presence of whole blood would be expected to yield more meaningful results. Blood contains biological components, such as proteases, antibody, and blood cells, which can affect the nature of a compound. For example, red blood cells and plasma proteins are known to affect the pharmacokinetics of drugs such as the anti-cancer agents docetaxel and gemcitabine (8,9). Vaidyanathan et al. (43) also reported that the cardioprotective drug, dexrazoxane, inhibits binding of the anti-cancer agent, doxorubicin, to red blood cells and that this interaction alters the pharmacokinetics of doxorubicin, and Clarke et al. (4) used an in vitro whole blood assay to study the binding affinity of a surrogate anti-CD11a monoclonal antibody to blood components. In addition, leukocytes produce a cytochrome P450 isoform (CYP2E1) that is involved in drug biotransformation (3). Thus, identifying and studying drugs in the presence of whole blood or blood components can offer a unique advantage over assays using cells in monoculture.

For studies on leukemia therapeutics, the cell line HL-60 is used as a standard target cell line. HL-60 cells were originally isolated from a 36-year-old female patient with acute promyelocytic leukemia (13). Testing the efficacy of anti-leukemia therapeutics against HL-60 cells in whole blood or other biological material is currently a challenge due to the inefficiency in differentiating the viability of HL-60 cells from other cells. Thus there remains a need to develop an efficient screen for anti-leukemia therapeutics and facilitate preclinical studies on a highly specific bacterial leukotoxin as a novel anti-leukemia therapeutic agent.

Accordingly, a stable bioluminescent HL-60 cell line whose viability can be rapidly and effectively determined in the presence of whole blood and live animals has now been developed along with an assay that allows for the rapid determination of the activity of a given drug against a leukemic cells either taken from a patient or derived from a cell line. The assay is carried out in the presence of whole blood or serum. This quantitative assay can screen thousands of drugs at a time or multiple concentrations of a drug in a 96- or 384-well format.

Screens for compounds and proteins with anti-cancer activity employ viability assays using relevant cancer cell lines. For leukemia studies, the human leukemia cell line, HL-60, is often used as a model system. To facilitate the discovery and investigation of anti-leukemia therapeutics under physiological conditions, HL-60 cells have been engineered that stably express firefly luciferase and produce light. Bioluminescent HL-60luc cells could be rapidly detected in whole blood with a sensitivity of approximately 1000 viable cells. Treatment of HL-60luc cells with a bacterial leukocyte-specific toxin or the drug chlorambucil revealed that the bioluminescent viability assay is more sensitive than the trypan blue dye exclusion assay. HL-60luc cells administered intraperitoneally (i.p) or intravenously (i.v.) were visualized in living mice using an in vivo imaging system (IVIS). The rapidity and ease of detecting HL-60luc cells in biological fluid indicates that this cell line can be used in high throughput screens for the identification of drugs with anti-leukemia activity under physiological conditions.

Other important objects and features of the invention will be apparent from the following description of the invention taken in connection with the accompanying drawings in which:

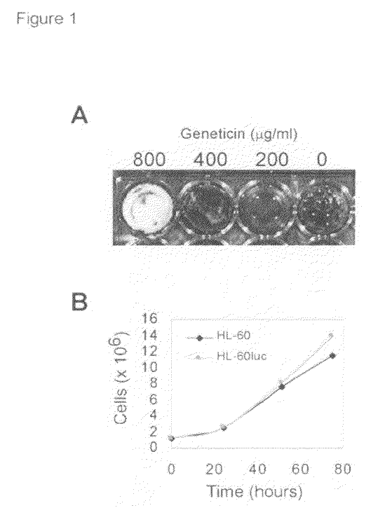

FIG. 1 shows the construction of a stable luciferase-expressing HL-60 cell line wherein (A) HL-60 cells were transfected with pMPI and then grown in wells with different concentrations of geneticin. Bioluminescence was detected with the IVIS 50 instrument and (B) Growth curves for parental HL-60 and engineered HL-60luc cells. Cells were grown in RPMI as described and viable cells were counted with the ViCELL cell counter.

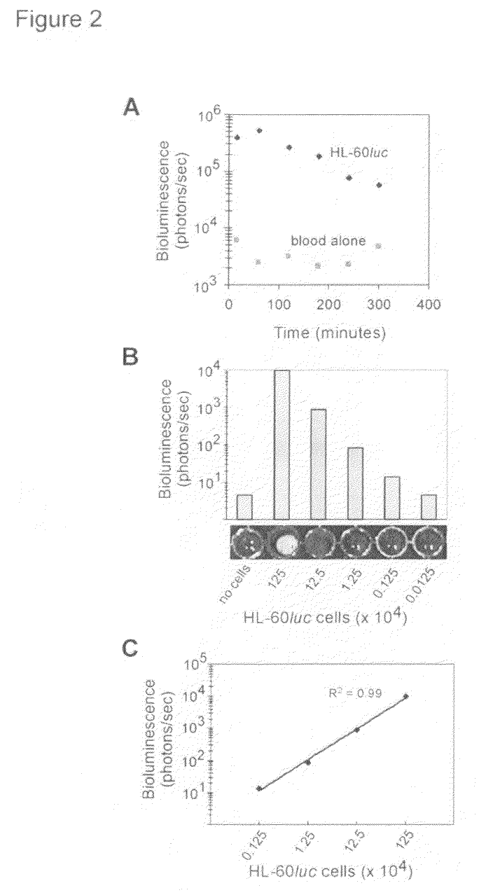

FIG. 2 shows the detection of HL-60luc cells in whole blood including (A) Kinetics of BL over time. HL-60luc cells were mixed with blood and luciferin and then imaged with the IVIS 50 instrument at the indicated time points. The observed pattern was highly reproducible. (B) Detection limit of HL-60luc cells. Cells were mixed with blood and luciferin and then incubated for one hour before imaging. (C) The number of HL-60luc cells shows a linear correlation with BL.

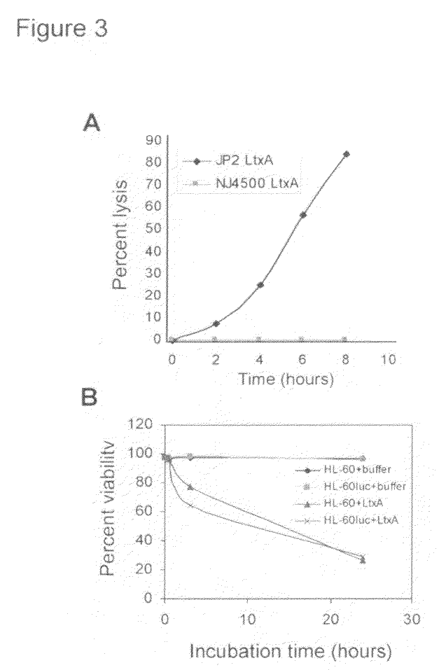

FIG. 3 shows effects of LtxA on cells. (A) Lysis of human red blood cells by LtxA from two different strains of A. actinomycetemcomitans. (B) HL-60 and HL-60luc cells are equally sensitive to killing by LtxA from strain NJ4500. Assays were performed in RPMI medium and viability was determined using the trypan blue dye exclusion assay.

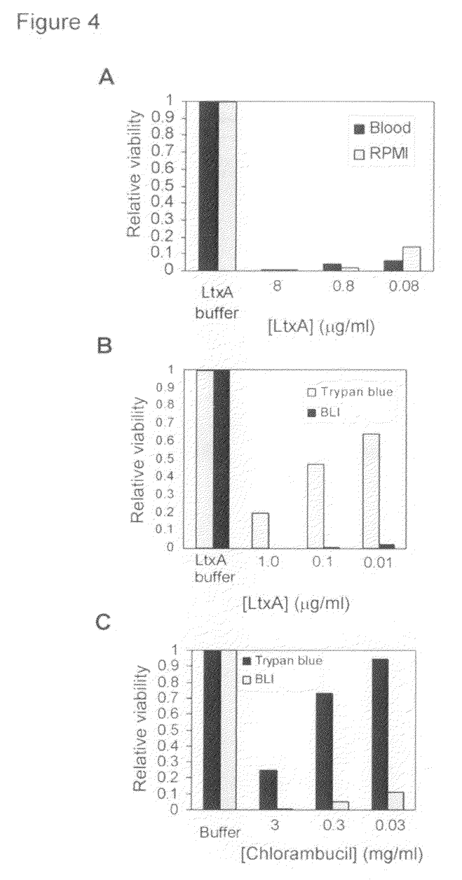

FIG. 4 shows the cytotoxicity of LtxA and chlorambucil. (A) Activity of LtxA against HL60luc cells in whole human blood and RPMI medium. Viability was measured using BL. (B) Comparison between BL and trypan blue as viability assays for LtxA-mediated cytotoxicity. Cells were incubated in RPMI medium with LtxA or buffer for 4 (??) hours and viability was determined. (C) Comparison between BL and trypan blue as viability assays for chlorambucil-mediated cytotoxicity. Cells were incubated in RPMI medium with chlorambucil or buffer for 24 hours and viability was determined

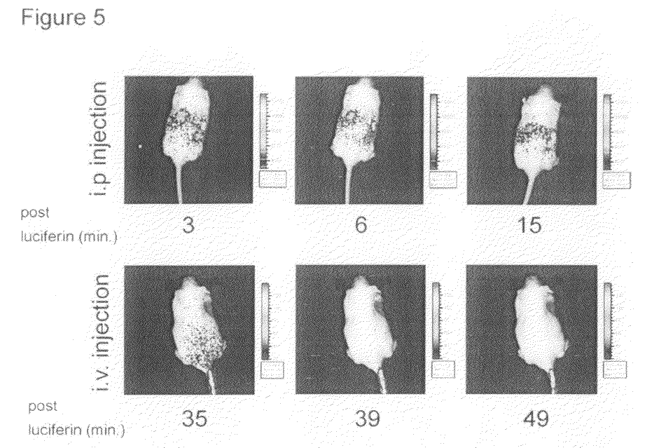

FIG. 5 shows bioluminescent imaging of HL-60luc cells in living mice. Swiss-Webster mice were anesthesized with XXX and injected with 10⁶ HL-60luc cells intraperitoneally (i.p.; top) or intravenously (i.v.; bottom) and followed by luciferin i.p. Mice were imaged with the IVIS 50 instrument at different times post luciferin injection. The scale on the right of each image indicates surface radiance (photons/second/cm²/steradian).

In vivo bioluminescence imaging (BLI) is a technology that allows visualization of live bioluminescent cells (mammalian, bacterial, viruses) in complex biological material and living animals (24,31). Firefly luciferase has been used extensively in reporter systems and its expression can be measured quantitatively using a luminometer or highly sensitive charge coupled device (CCD) camera. Rocchetta et al. (32) found that the CCD camera was approximately 25 times more sensitive than a luminometer, and so the IVIS 50 imaging system (Xenogen, Alameda, Calif.) was used for the work presented here. Luciferase reacts with its substrate, luciferin, to produce oxyluciferin and light (11). Because ATP and oxygen are required for the reaction, photon production has been used as a quantitative measurement of cellular viability (14). Animal studies have demonstrated a strong correlation between the abundance of emitted photons and number of cells present in a given tissue or animal (5,11).

In general, the field of oncology has utilized BLI extensively to study the effects of anti-cancer therapy in vivo (15,23). However, application of BLI to study hematologic malignancies has been limited (6,22,44), and to date, there are no bioluminescent hematologic cell lines commercially available (Xenogen Corp., Alameda, Calif.). Validation of BLI in preclinical models has been carried out using currently available methods and evidence indicates that BLI has excellent sensitivity and offers unique advantages (5,25,31,33). For example, non-invasive BLI allows visualization of cells temporally and spatially, thus permitting small changes in cell number and localization to be detected over time (24,31). In addition, animals need not be sacrificed at each sampling time point, decreasing the number of animals that are required for an experiment and minimizing inconsistency from animal-to-animal variations. A bioluminescent HL-60 cell line has been engineered that can be visualized in whole human blood and living mice and whose viability can be rapidly determined. A WBC-specific bacterial toxin has been shown to be active in blood. The engineered HL-60luc cells of the invention behave similar to the parental HL60 cell line. The BLI signal peaked approximately one hour following the addition of luciferin but remained relatively high for several hours. This type of in vitro kinetics where an early peak in luminescence is followed by a slow decline is consistent with other BLI cell lines. The detection limit of 1000 viable cells is also consistent with other reports (35,36). Because human blood contains plasma proteins, such as antibody and proteases, and other cells, that may affect the activity, availability, or stability of a drug, the anti-leukemia assays with HL-60luc cells in the presence of blood can yield more physiological results than with buffer or growth media alone.

There is a significant difference between the sensitivity of BLI and the trypan blue dye. exclusion assay. For a cell to be detected as nonviable with the trypan blue assay, the dye must enter the cytoplasm of the cell. Trypan blue is a relatively large molecule (mw 960.8) and while many cells may be metabolically dead, their membranes could be sufficiently intact to exclude the dye to appear viable. In contrast, BLI detects killing sooner because ATP is no longer available in a metabolically dead cell. The results are in strong agreement with Kuzmits et al. (17) who found that an ATP/bioluminescent assay with HL-60 cells indicated nearly complete killing after a 24 hour incubation with 5.7 μmol/l doxorubicin, while the trypan blue assay indicated almost no killing after 48 hours with the same drug concentration. Furthermore, Petty et al. (30) reported that a bioluminescent ATP assay could detect as few as 1500 viable cells/well while the MTT assay could not detect less than 25,000 cells/well.

Bacterial toxins have been investigated for their anti-cancer therapeutic potential for many years. Several widely-studied toxins include diphtheriae toxin (DT) and Pseudomonas exotoxin A (PE) (16). To increase the specificity of these toxins, their toxic domains are often fused to other molecules that target the toxin to certain cell types. For example, ONTAK, a recently approved drug used to treat cutaneous T-cell lymphoma, is a fusion molecule of DT and IL-2 (10,26).

The oral bacterium A. actinomycetemcomitans produces a 113 kDa protein toxin, leukotoxin (LtxA), which kills only blood cells of humans and Old World Primates (37-39). Furthermore, a strain has been identified whose purified LtxA does not lyse RBCs. LtxA binds to LFA-1 on host cells (19) and destroys cells by apoptosis or necrosis (18). Because LtxA already has specificity towards WBCs, it has been proposed that the protein might serve as an effective targeted therapy for hematologic malignancies. In addition, the toxin kills host cells by disruption of the cell membrane (18) and therefore represents a mechanism of action that is different from other chemotherapeutic agents. In an effort to evaluate the therapeutic potential of LtxA, its toxic effects against HL-60luc cells in blood were examined. The toxin remains highly active in human blood and kills HL60luc cells as efficiently as in RPMI medium. In addition, bone marrow progenitor cell proliferation assays indicate that LtxA is active toward myeloid progenitor cells and has an IC₅₀ value in the low ng/ml range. Preliminary studies also suggest that LtxA is active mice and does not display toxicity when injected at high doses into mice.

With the ability to rapidly determine HL-60luc cell viability in the presence of biological fluids, it is expected that it would be possible to efficiently screen thousands of different compounds at a time for anti-leukemia activity. Assays could be performed in 96- or 384-well dishes in the presence of physiologically-relevant sample such as blood, plasma, or hepatocytes. Indeed, an important preclinical screen to study drug biotransformation is performed in the presence of hepatic material, such as human liver microsomes, human liver cytosol fractions, and hepatocytes (3). HL-60luc cells could be used in high-throughput hepatic screens for drugs with anti-leukemia bioactivity. In addition to using HL-60luc cells for drug discovery, the behavior of a known drug or combination of drugs in the presence of blood samples from different leukemia patients could be determined. For example, neutralizing antibody in a patient's blood against a potential drug might allow a clinician to exclude the drug from the therapeutic regimen. Excluding an otherwise ineffective drug might greatly reduce unwanted side effects. Indeed several studies have shown a correlation between in vitro chemosensitivity of tumor cells and therapy outcome (34,42). Such correlations could allow the development of assay-directed individualized chemotherapy regimens. Thus the assay of the invention can be used in the following ways:

1) Screening novel drugs for anti-leukemia/cancer activity.

2) Determine the best drug dosage for a leukemia/cancer patient.

3) Determine which drug might be most effective for a leukemia/cancer patient.

### Experimental

Cells and growth conditions. HL-60 cells were obtained from American Type Culture Collection (ATCC) and maintained in RPMI+10% fetal bovine serum (FBS) (Invitrogen, Carlsbad, Calif.) at 37° C.+5% CO₂. Escherichia coli was grown in LB medium at 37° C. A. actinomycetemcomitans strains were grown in AAGM at 37° C.+10% CO₂ as previously described (12).

DNA manipulations. The luciferase-encoding plasmid for transfecting HL-60 cells was constructed by cloning luciferase gene from pGL3 (Promega, Madison, Wis.) into the geneticin resistance gene-containing plasmid pCI-neo (Promega, Madison, Wis.). Both plasmids were digested with BglII and XbaI and the Neo-containing fragment was then ligated to the pGL3 fragment that contained the luciferase gene. The mixture was transformed into E. coli and the bacteria were selected on LB+carbenecillin (50 μg/ml). Plasmid from bacteria was prepared using the plasmid miniprep kit (Qiagen, Valencia, Calif.). The new plasmid, encoding both luciferase and geneticin, was designated pMP1.

The plasmid, pMP1, was transfected into HL-60 cells by electroporation. Briefly, 10⁶ cells were resuspended in 400 μl electroporation buffer (20 mM HEPES pH 7.0, 137 mM NaCl, 5 mM KCl, 0.7 mM Na₂HPO₄, 6 mM glucose, 0.1 mM β-mercaptoethanol). Plasmid pMP1 was added at a concentration of 12.5 μg/ml and the mixture was incubated for 5 minutes on ice. The mixture was added to a cuvette and a pulse of 380 V was administered. Five ml of fresh RPMI medium was added to the cells and they were grown for 24 hours before geneticin was added.

### Preparation of Cytotoxic Agents

Bacterial leukotoxin (LtxA) was purified from A. actinomycetemcomitans as previously described (7). LtxA was stored in 100 μl aliquots at −80° C. until used. A stock solution of chlorambucil (Sigma, St. Louis, Mo.) was prepared by dissolving 30 mg into 1 ml of DMSO. The drug was freshly prepared prior to each experiment. Bioluminescent imaging (BLI). For detection of bioluminescence (BL) from cultured HL-60luc cells, 200 μl of cells were mixed with 1 μl luciferin (15 mg/ml) and then imaged with the IVIS 50 imaging system (Xenogen Corp., Alameda, Calif.). For animal studies, Swiss Webster mice were first injected with 10⁶ HL-60luc cells (resuspended in PBS) or PBS control intraperitoneally (i.p.) or intravenously (i.v.). Mice were then anesthesized with acepromazine (0.3 mg/40 g, i.p.) and a rodent cocktail [ketamine (20 mg/ml) and xylazine (2.5 mg/ml)] (0.1 ml/25 g, i.p.). Luciferin was then injected (150 mg/kg) i.p and the mice were imaged with the IVIS 50 instrument at different times. Images were analyzed using the Living Image Software (Xenogen Corp., Alameda, Calif.).

### Results

Construction of a Stable HL-60 Luciferase-Expressing Cell Line. To generate an HL-60 cell line that stably expresses luciferase, a plasmid was constructed by cloning the luciferase gene from pGL3 into the geneticin resistance gene-containing plasmid pCIneo. The modified plasmid, pMP1, was then electroporated into HL-60 cells (obtained from ATCC) and grown under geneticin selection. When geneticin was included in the growth medium to select for the plasmid, bioluminescence (BL) was observed, indicating that cells received the luciferase-encoding plasmid. FIG. 1A shows HL-60 cells that were transfected with pMP 1 and then grown in wells with different concentrations of geneticin. Bioluminescence was detected with the IVIS 50 instrument. Cells were grown for 8 weeks longer to allow the generation of stable clones. After 8 weeks, geneticin selection was removed to determine if the luciferase-encoding gene had successfully integrated into the genome. Even after growing cells for many generations without selection, the HL-60 cells still emitted light, suggesting that stable transfectants had been obtained

To continue studies, a homogeneous population of cells derived from a single stable clone was isolated by performing minimal dilutions with stable transfectants. Cells were diluted to approximately one cell/well in a 96-well dish and then examined microscopically to exclude wells that received more than one cell. Dishes were further incubated and then imaged with the IVIS 50 instrument. Cells were transferred to larger dishes, grown and then and saved in liquid nitrogen. Viability of these saved cells was greater than 90%.

An important property for BLI studies is photon flux per cell (photons/second/cell). The flux/cell for one specific clone that was used in all subsequent assays described here was calculated. The calculated value of 16 photons/second/cell is consistent with values obtained from other engineered cell lines (Xenogen Corp., Alameda, Calif.). It is believed that this is the first HL-60 cell line that has been engineered to stably express luciferase.

To confirm that the engineered HL-60 cells maintain basic growth characteristics, growth studies were performed comparing HL-60luc cells to parental HL-60 cells. Cells were grown in RPMI with 10% FBS and then counted with a Vi-CELL cell viability analyzer (Beckman Coulter, Inc., Miami, Fla.). Growth curve experiments in RPMI for the two cell lines indicated that HL-60luc cells behave like the parental cell line. FIG. 1B shows growth curves for parental HL-60 and engineered HL-60luc cells. Cells were grown in RPMI as described and viable cells were counted with the ViCELL cell counter.

Detection of HL-60luc in blood. To determine the kinetics of bioluminescence in blood, 6×10⁵ HL-60luc cells were mixed with human peripheral blood and luciferin was added to the mixture. BL was then measured over time as photons/second from each sample. FIG. 2A shows the kinetics of BL over time. HL-60luc cells were mixed with blood and luciferin and then imaged with the IVIS 50 instrument at the indicated time points. The observed pattern was highly reproducible. The signal peaked at one hour and was approximately 200 times greater than the background signal from blood alone. FIG. 2A. These results were highly reproducible and a similar pattern was obtained when the same experiment was performed in RPMI. BL values in RPMI were approximately two-fold greater than in blood likely due to light absorption by the blood.

The sensitivity of detection in blood was then determined. Different numbers of HL-60luc cells were mixed with blood (200 μl total) and luciferin was added to each sample. The mixtures were incubated at 37° C. for one hour and BL was measured. FIG. 2B shows detection limit of HL-60luc cells. Cells were mixed with blood and luciferin and then incubated for one hour before imaging. Approximately 1000 cells could be detected above the background level of the blood alone. The signal emitted from the highest number of cells tested (1.25×10⁶) was approximately 2000 times greater than blood alone. The BL signal correlated strongly with cell number. FIG. 2C shows that the number of HL-60luc cells shows a linear correlation with BL.

Sensitivity of HL-60luc cells to a bacterial toxin. The gram negative bacterium, A. actinomycetemcomitans, produces leukotoxin (LtxA), a protein toxin that kills specifically white blood cells from humans and Old World Primates (37-39) and red blood cells (1). Examination of LtxA from a strain of A. actinomycetemcomitans, NJ4500, revealed that this purified protein does not lyse erythrocytes in vitro compared to LtxA from the standard strain, JP2. FIG. 3A shows the lysis of human red blood cells by LtxA from two different strains of A. actinomycetemcomitans. Because erythrocyte lysis would be an undesirable property for a chemotherapeutic agent, studies here employ LtxA from NJ4500.

To determine if HL-60luc cells are equally sensitive to LtxA as parental HL-60 cells, cell killing was assayed by LtxA in RPMI. HL-60 cells were mixed with LtxA and viability was measured with the trypan blue dye exclusion assay using the Vi-CELL instrument. LtxA had an equal toxic effect on both cell lines. FIG. 3B shows that HL-60 and HL-60luc cells are equally sensitive to killing by LtxA from strain NJ4500. Assays were performed in RPMI medium and viability was determined using the trypan blue dye exclusion assay. This result was highly reproducible. Thus, the HL-60luc cell line is similar to the parental HL-60 cell line for it sensitivity to a bacterial toxin.

LtxA activity in whole blood. To determine if LtxA is active in whole blood and retains its ability to kill HL-60 cells, HL-60luc cells were resuspended in blood or RPMI and different concentrations of purified LtxA or LtxA buffer was added to the HL-60luc-blood mixture and incubated at 37° C. for 4 hours. BLI was then measured and relative viabilities were determined comparing experimental values to the buffer-containing sample. LtxA was highly active in whole blood against HL-60luc cells and this activity was similar to that seen in RPMI. FIG. 4A shows the activity of LtxA against HL60luc cells in whole human blood and RPMI medium. Viability was measured using BL.

The sensitivity of BL to trypan blue as an assay for cell viability was also compared. Luminescence was significantly more sensitive than trypan blue. FIG. 4B shows the comparison between BL and trypan blue as viability assays for LtxA-mediated cytotoxicity. Cells were incubated in RPMI medium with LtxA or buffer for 4 hours and viability was determined. Nearly complete cell killing was observed with leukotoxin concentrations as low as 10 ng/ml using BL values. In contrast, trypan blue revealed that only 35% killing had occurred at this concentration.

To determine if the difference in detection limit between the two methods was specific for LtxA-mediated cytotoxicity, another compound, chlorambucil, was used to induce cell death. Chlorambucil alkylates DNA and induces apoptosis (2,21) and therefore represents a mechanism of killing different from that of LtxA. For chlorambucil, it was also observed that BLI was a more sensitive assay than trypan blue for detecting viability. FIG. 4C shows the comparison between BL and trypan blue as viability assays for chlorambucil-mediated cytotoxicity. Cells were incubated in RPMI medium with chlorambucil or buffer for 24 hours and viability was determined. At a chlorambucil concentration of 0.03 mg/ml, BLI revealed approximately 90% cell death after 24 hours while trypan blue revealed essentially no killing (FIG. 4C).

Visualization of HL-60luc in mice. Mouse models for human leukemia utilize HL-60 cells that are injected either i.p. (28) or i.v. (40,41). To determine if the HL-60luc cells could be visualized in living mice, approximately 10⁶ HL-60luc cells were injected i.p. or tail i.v. FIG. 5 shows Swiss-Webster mice that were anesthesized with XXX and injected with 10⁶ HL-60luc cells intraperitoneally (i.p.; top) or intravenously (i.v.; bottom) and followed by luciferin i.p. Mice were imaged with the IVIS 50 instrument at different times post luciferin injection. The scale on the right of each image indicates surface radiance (photons/second/cm²/steradian). Luciferin was administered immediately following injection of cells and the animals were imaged with the IVIS 50 instrument. The cells could be detected with a 2-3 minute exposure when administered by either route. The signal was greatest for i.p.-injected cells immediately following injection while the signal for i.v.-injected cells peaked approximately 35 minutes post luciferin (FIG. 5). Interestingly, the signal observed for i.v. injection follows the path of the tail vein and then dissipates as the cells become diluted through other blood vessels. Thus, HL-60 cells can be visualized in a living animal at concentrations normally used for the SCID mouse model for human leukemia.

### REFERENCES

1. Balashova, N. V., J. A. Crosby, L. Al Ghofaily and S. C. Kachlany. 2006. Leukotoxin confers beta-hemolytic activity to Actinobacillus actinomycetemcomitans. Infect Immun 74:2015-2021.
2. Begleiter, A., M. Mowat, L. G. Israels and J. B. Johnston. 1996. Chlorambucil in chronic lymphocytic leukemia: mechanism of action. Leuk Lymphoma 23:187-201.
3. Brandon, E. F., C. D. Raap, I. Meijerman, J. H. Beijnen and J. H. Schellens. 2003. An update on in vitro test methods in human hepatic drug biotransformation research: pros and cons. Toxicol Appl Pharmacol 189:233-246.
4. Clarke, J., W. Leach, S. Pippig, A. Joshi, B. Wu, R. House and J. Beyer. 2004. Evaluation of a surrogate antibody for preclinical safety testing of an antiCD11a monoclonal antibody. Regul Toxicol Pharmacol 40:219-226.
5. Contag, C. H. and M. H. Bachmann. 2002. Advances in in vivo bioluminescence imaging of gene expression. Annu Rev Biomed Eng 4:235-260.
6. Cooper, L. J., Z. Al-Kadhimi, L. M. Serrano, T. Pfeiffer, S. Olivares, A. Castro, W. C. Chang, S. Gonzalez, D. Smith, S. J. Forman and M. C. Jensen. 2005. Enhanced antilymphoma efficacy of CD 19-redirected influenza MP1 specific CTLs by cotransfer of T cells modified to present influenza MP1. Blood 105:1622-1631.
7. Diaz, R., L. A. Ghofaily, J. Patel, N. V. Balashova, A. C. Freitas, I. Labib and S. C. Kachlany. 2006. Characterization of leukotoxin from a clinical strain of Actinobacillus actinomycetemcomitans. Microb Pathog 40:48-55.
8. Dumez, H., G. Guetens, G. De Boeck, M. S. Highley, E. A. de Bruijn, A. T. van Oosterom and R. A. Maes. 2005. In vitro partition of docetaxel and gemcitabine in human volunteer blood: the influence of concentration and gender. Anticancer Drugs 16:885-891.
9. Dumez, H., W. H. Reinhart, G. Guetens and E. A. de Bruijn. 2004. Human red blood cells: rheological aspects, uptake, and release of cytotoxic drugs. Crit Rev Clin Lab Sci 41:159-188.
10. Duvic, M., T. M. Kuzel, E. A. Olsen, A. G. Martin, F. M. Foss, Y. H. Kim, P. W. Heald, P. Bacha, J. Nichols and A. Liepa. 2002. Quality-of-life improvements in cutaneous T-cell lymphoma patients treated with denileukin diftitox (ONTAK). Clin Lymphoma 2:222-228.
11. Edinger, M., Y. A. Cao, Y. S. Hornig, D. E. Jenkins, M. R. Verneris, M. H. Bachmann, R. S. Negrin and C. H. Contag. 2002. Advancing animal models of neoplasia through in vivo bioluminescence imaging. Eur J Cancer 38:2128-2136.
12. Fine, D. H., D. Furgang, H. C. Schreiner, P. Goncharoff, J. Charlesworth, G. Ghazwan, P. Fitzgerald-Bocarsly and D. H. Figurski. 1999. Phenotypic variation in Actinobacillus actinomycetemcomitans during laboratory growth: implications for virulence. Microbiology 145 (Pt6):1335-1347.
13. Gallagher, R., S. Collins, J. Trujillo, K. McCredie, M. Ahearn, S. Tsai, R. Metzgar, G. Aulakh, R. Ting, F. Ruscetti and R. Gallo. 1979. Characterization of the continuous, differentiating myeloid cell line (HL-60) from a patient with acute promyelocytic leukemia. Blood 54:713-733.
14. Hill, P. J., G. S. Stewart and P. E. Stanley. 1993. Bioluminescence and chemiluminescence literature. Luciferase reporter genes—lux and luc. Part 2. J Biolumin Chemilumin 8:267-291.
15. Hollingshead, M. G., C. A. Bonomi, S. D. Borgel, J. P. Carter, R. Shoemaker, G. Melillo and E. A. Sausville. 2004. A potential role for imaging technology in anticancer efficacy evaluations. Eur J Cancer 40:890-898.
16. Jain, K. K. 2001. Use of bacteria as anticancer agents. Expert Opin Biol Ther 1:291-300.
17. Kuzmits, R., H. Rumpold, M. M. Muller and G. Schopf. 1986. The use of bioluminescence to evaluate the influence of chemotherapeutic drugs on ATP-levels of malignant cell lines. J Clin Chem Clin Biochem 24:293-298.
18. Lally, E. T., R. B. Hill, I. R. Kieba and J. Korostoff. 1999. The interaction between RTX toxins and target cells. Trends Microbiol 7:356-361.
19. Lally, E. T., I. R. Kieba, A. Sato, C. L. Green, J. Rosenbloom, J. Korostoff, J. F. Wang, B. J. Shenker, S. Ortlepp, M. K. Robinson and P. C. Billings. 1997. RTX toxins recognize a beta2 integrin on the surface of human target cells. J Biol Chem 272:30463-30469.
20. Liu, T. F., J. O. Urieto, J. E. Moore, M. S. Miller, A. C. Lowe, A. Thorburn and A. E. Frankel. 2004. Diphtheria toxin fused to variant interleukin-3 provides enhanced binding to the interleukin-3 receptor and more potent leukemia cell cytotoxicity. Exp Hematol 32:277-281.
21. Masta, A., P. J. Gray and D. R. Phillips. 1995. Nitrogen mustard inhibits transcription and translation in a cell free system. Nucleic Acids Res 23:3508-3515.
22. Mitsiades, C. S., N. S. Mitsiades, C. J. McMullan, V. Poulaki, R. Shringarpure, M. Akiyama, T. Hideshima, D. Chauhan, M. Joseph, T. A. Libermann, C. Garcia-Echeverria, M. A. Pearson, F. Hofmann, K. C. Anderson and A. L. Kung. 2004. Inhibition of the insulin-like growth factor receptor-1 tyrosine kinase activity as a therapeutic strategy for multiple myeloma, other hematologic malignancies, and solid tumors. Cancer Cell 5:221-230.
23. Mocanu, J. D., E. H. Moriyama, M. C. Chia, J. H. Li, K. W. Yip, D. P. Huang, C. Bastianutto, B. C. Wilson and F. F. Liu. 2004. Combined in vivo bioluminescence and fluorescence imaging for cancer gene therapy. Mol Imaging 3:352-355.
24. Nogawa, M., T. Yuasa, S. Kimura, J. Kuroda, K. Sato, H. Segawa, A. Yokota and T. Maekawa. 2005. Monitoring luciferase-labeled cancer cell growth and metastasis in different in vivo models. Cancer Lett 217:243-253.
25. Nyati, M. K., Z. Symon, E. Kievit, K. J. Dornfeld, S. D. Rynkiewicz, B. D. Ross, A. Rehemtulla and T. S. Lawrence. 2002. The potential of 5-fluorocytosine/cytosine deaminase enzyme prodrug gene therapy in an intrahepatic colon cancer model. Gene Ther 9:844-849.
26. Olsen, E., M. Duvic, A. Frankel, Y. Kim, A. Martin, E. Vonderheid, B. Jegasothy, G. Wood, M. Gordon, P. Heald, A. Oseroff, L. Pinter-Brown, G. Bowen, T. Kuzel, D. Fivenson, F. Foss, M. Glode, A. Molina, E. Knobler, S. Stewart, K. Cooper, S. Stevens, F. Craig, J. Reuben, P. Bacha and J. Nichols. 2001. Pivotal phase III trial of two dose levels of denileukin diftitox for the treatment of cutaneous T-cell lymphoma. J Clin Oncol 19:376-388.
27. Pereira, C., J. Damen, A. Eaves and E. Clarke. 2004. Toxicity testing: What bone marrow can tell us. BioProcess International 2:70-75.
28. Perentesis, J. P., R. Gunther, B. Waurzyniak, Y. Yanishevski, D. E. Myers, O. Ek, Y. Messinger, Y. Shao, L. M. Chelstrom, E. Schneider, W. E. Evans and F. M. Uckun. 1997. In vivo biotherapy of HL-60 myeloid leukemia with a genetically engineered recombinant fusion toxin directed against the human granulocyte macrophage colony-stimulating factor receptor. Clin Cancer Res 3:2217-2227.
29. Pessina, A., I. Malerba and L. Gribaldo. 2005. Hematotoxicity testing by cell clonogenic assay in drug development and preclinical trials. Curr Pharm Des 11:1055-1065.
30. Petty, R. D., L. A. Sutherland, E. M. Hunter and I. A. Cree. 1995. Comparison of MTT and ATP-based assays for the measurement of viable cell number. J Biolumin Chemilumin 10:29-34.
31. Rehemtulla, A., L. D. Stegman, S. J. Cardozo, S. Gupta, D. E. Hall, C. H. Contag and B. D. Ross. 2000. Rapid and quantitative assessment of cancer treatment response using in vivo bioluminescence imaging. Neoplasia 2:491-495.
32. Rocchetta, H. L., C. J. Boylan, J. W. Foley, P. W. Iversen, D. L. LeTourneau, C. L. McMillian, P. R. Contag, D. E. Jenkins and T. R. Parr, Jr. 2001. Validation of a noninvasive, real-time imaging technology using bioluminescent Escherichia coli in the neutropenic mouse thigh model of infection. Antimicrob Agents Chemother 45:129-137.
33. Sadikot, R. T., L. J. Wudel, D. E. Jansen, J. P. Debelak, F. E. Yull, J. W. Christman, T. S. Blackwell and W. C. Chapman. 2002. Hepatic cryoablationinduced multisystem injury: bioluminescent detection of NF-kappaB activation in a transgenic mouse model. J Gastrointest Surg 6:264-270.
34. Samson, D. J., J. Seidenfeld, K. Ziegler and N. Aronson. 2004. Chemotherapy sensitivity and resistance assays: a systematic review. J Clin Oncol 22:3618-3630.
35. Sarraf-Yazdi, S., J. Mi, M. W. Dewhirst and B. M. Clary. 2004. Use of in vivo bioluminescence imaging to predict hepatic tumor burden in mice. J Surg Res 120:249-255.
36. Sweeney, T. J., V. Mailander, A. A. Tucker, A. B. Olomu, W. Zhang, Y. Cao, R. S. Negrin and C. H. Contag. 1999. Visualizing the kinetics of tumor-cell clearance in living animals. Proc Natl Acad Sci USA 96:12044-12049.
37. Taichman, N. S., R. T. Dean and C. J. Sanderson. 1980. Biochemical and morphological characterization of the killing of human monocytes by a leukotoxin derived from Actinobacillus actinomycetemcomitans. Infect Immun 28:258-268.
38. Taichman, N. S., D. L. Simpson, S. Sakurada, M. Cranfield, J. DiRienzo and J. Slots. 1987. Comparative studies on the biology of Actinobacillus actinomycetemcomitans leukotoxin in primates. Oral Microbiol Immunol 2:97-104.
39. Taichman, N. S. and J. M. Wilton. 1981. Leukotoxicity of an extract from Actinobacillus actinomycetemcomitans for human gingival polymorphonuclear leukocytes. Inflammation 5:1-12.
40. Tomkinson, B., R. Bendele, F. J. Giles, E. Brown, A. Gray, K. Hart, J. D. LeRay, D. Meyer, M. Pelanne and D. L. Emerson. 2003. OSI-211, a novel liposomal topoisomerase I inhibitor, is active in SCID mouse models of human AML and ALL. Leuk Res 27:1039-1050.
41. Uckun, F. M. 1996. Severe combined immunodeficient mouse models of human leukemia. Blood 88:1135-1146.
42. Ugurel, S., D. Schadendorf, C. Pfohler, K. Neuber, A. Thoelke, J. Ulrich, A. Hauschild, K. Spieth, M. Kaatz, W. Rittgen, S. Delorme, W. Tilgen and U. Reinhold. 2006. In vitro drug sensitivity predicts response and survival after individualized sensitivity-directed chemotherapy in metastatic melanoma: a multicenter phase II trial of the Dermatologic Cooperative Oncology Group. Clin Cancer Res 12:5454-5463.
43. Vaidyanathan, S. and M. Boroujerdi. 2000. Interaction of dexrazoxane with red blood cells and hemoglobin alters pharmacokinetics of doxorubicin. Cancer Chemother Pharmacol 46:93-100.
44. Walensky, L. D., A. L. Kung, I. Escher, T. J. Malia, S. Barbuto, R. D. Wright, G. Wagner, G. L. Verdine and S. J. Korsmeyer. 2004. Activation of apoptosis in vivo by a hydrocarbon-stapled BH3 helix. Science 305:1466-1470.
45. Ward, T. H., S. Danson, A. T. McGown, M. Ranson, N. A. Coe, G. C. Jayson, J. Cummings, R. H. Hargreaves and J. Butler. 2005. Preclinical evaluation of the pharmacodynamic properties of 2,5-diaziridinyl-3-hydroxymethyl-6-methyl-1,4-benzoquinone. Clin Cancer Res 11:2695-2701.

1. An assay for the determination of the activity of a drug against leukemic cells wherein the assay is carried out in the presence of whole blood or serum.
